(19) United States
(12) Patent Application Publication
(10) Pub. No.: US 2021/0015638 A1
(43) Pub. Date: Jan. 21, 2021

(54) SINGLE SPOOL VALVE/MOTOR CONTROL FOR BIDIRECTIONAL MOVEMENT OF HYDRAULIC PROSTHETIC AND HALL EFFECT SENSOR FOR FORCE MEASUREMENT

(71) Applicant: College Park Industries, Inc., Warren, MI (US)

(72) Inventors: Aaron Taszreak, China, MI (US); Jacob Drews, Washington, MI (US); Kevin L'Heureux, Lincoln Park, MI (US)

(21) Appl. No.: 16/930,883

(22) Filed: Jul. 16, 2020

Related U.S. Application Data
(60) Provisional application No. 62/875,626, filed on Jul. 18, 2019.

(51) Int. Cl. A61F 2/68; F16K 11/076; F15B 15/20

(52) U.S. Cl. CPC A61F 2/68; A61F 2002/745; F15B 15/20; F16K 11/076

(57) ABSTRACT

A hydraulic prosthetic device has upper and lower portions and a hydraulic cylinder coupled thereto for damping relative motion. The cylinder has a movable piston and a first and second chamber. A three-way valve includes a housing and a movable portion cooperating to define a hydraulic circuit. The movable portion has a master port, an inlet port and an outlet port, and the housing has an inlet opening, an outlet opening, and a master opening each positioned to fluidly communicate with the respective port. The inlet port and/or the inlet opening, and the outlet port and/or the outlet opening are shaped such that movement of the movable portion of the valve relative to the housing varies a resistance to the fluid flow related to movement or the prosthetic portions in both directions.

### CROSS REFERENCE TO RELATED APPLICATIONS

This application claims priority to U.S. Provisional Patent Application Ser. No. 62/875,626, filed Jul. 18, 2019, the entire content of which is incorporated herein by reference.

### FIELD OF THE INVENTION

The present invention relates to control of bidirectional movement of a hydraulic prosthetic such as a prosthetic foot and to force measurement using Hall effect sensors.

### BACKGROUND OF THE INVENTION

In hydraulically dampened prosthetic devices, such as feet and knees, dampening characteristics typically are controlled and adjusted through the use of 2 valves, one for movement in one direction, such as plantarflexion of a foot, and the other for movement in the other direction, such as dorsiflexion of a foot. In microprocessor-controlled versions, motors are applied to the valves which by some predetermined states adjust the valves automatically depending on environmental conditions. This may add significant weight and size to the devices, which is undesired.

The traditional means of measuring forces on lower limb prosthetic devices is to use a strain gauge. Strain gauges are very thin and fragile components. Analog signal conditioning and significant gain are also used in this type of measurement. This all leads to a complex and fragile system for determining load. In the case of composite springs used in prosthetics, these bending members go through extreme strain with a high cycle count. The requirements of a gauge attached to these members may be extreme, and far exceed the ratings of such devices. The proper application of strain gauges to prosthetics is then a complex and challenging problem.

### SUMMARY OF THE INVENTION

An embodiment of the present invention provides a control for movement in first and second directions of a hydraulic prosthetic device using a single three-way valve. An example of movement in a first and second direction is plantarflexion and dorsiflexion of a hydraulic prosthetic foot. The present invention is also applicable to other prosthetic devices such as, but not limited to, a prosthetic knee, where movement is to be controlled in two directions.

The three-way valve controls the resistance to flow of hydraulic fluid due to movement in the first direction and in the second direction, and allows the resistance to both fluid flows to be adjusted by moving the single three-way valve. Ports and/or openings in the movable portion and housing of the valve are shaped such that movement of the movable portion relative to the housing varies the resistance to both flows. A motor may control movement of the movable portion, and a plurality of port opening pairs may be provided to allow changes in the relative flow resistances.

A first embodiment of a hydraulic prosthetic, with control of bidirectional movement, includes a prosthetic having an upper portion and a lower portion movably connected to the upper portion. A hydraulic cylinder has a movable piston and a first chamber and a second chamber, the hydraulic cylinder coupled to the upper and lower portions of the prosthetic such that movement of the lower portion relative to the upper portion in a first direction causes the first chamber to reduce in size and the second chamber to expand and movement of the lower portion relative to the upper portion in a second direction causes the first chamber to expand and the second camber to reduce in size. A three-way valve includes a housing and a movable portion that is movable relative to the housing, the housing and movable portion cooperating to define a hydraulic circuit. The movable portion has a master port, an inlet port and an outlet port, the master port being in fluid communication with the inlet and outlet ports. The housing has an inlet opening positioned to selectively fluidly communicate with the inlet port of the movable portion, an outlet opening positioned to selectively fluidly communicate with the outlet port of the movable portion, and a master opening positioned to fluidly communicate with the master port. A first fluid passage extends from the first chamber to the master opening, a second fluid passage extends from the second chamber to the inlet opening of the housing, and a third fluid passage extends from the second chamber to the outlet opening of the housing.

The valve and fluid passages cooperate to selectively define a first fluid circuit from the first chamber, through the first fluid passage, through the master opening and master port, to and through the outlet port and outlet opening, and through the third fluid passage to the second chamber of the hydraulic cylinder; and to selectively define a second fluid circuit from the second chamber, through the second fluid passage, through the inlet opening and the inlet port, to and through the master port and master opening and through the first fluid passage to the first chamber of the hydraulic cylinder. A first one-way valve is disposed in the first fluid circuit and is operable to allow fluid to flow substantially only in a direction from the first chamber to the second chamber. A second one-way valve is disposed in the second fluid circuit and is operable to allow fluid to flow substantially only in a direction from the second chamber to the first chamber. The inlet port and/or the inlet opening, and the outlet port and/or the outlet opening are shaped such that movement of the movable portion of the valve relative to the housing varies a resistance to the fluid flow in both the first and second fluid circuits.

In some examples, the prosthetic comprises a prosthetic foot, the upper portion being for connection to a leg and the lower portion being a lower foot portion, the movement in the first direction being dorsiflexion and the movement in the second direction being plantarflexion.

The hydraulic prosthetic may include a motor operable to move the movable portion of the valve relative to the housing for adjusting the resistance to fluid flow in the circuits. The three way valve may be a spool valve with the movable portion be rotatably movable relative to the housing. In some examples, the movable portion has an end surface and a circumferential side surface, the master port, inlet port, and outlet port being disposed in the side surface. In some examples, the inlet port and outlet port and are disposed on opposite sides of the movable portion.

In some examples, the inlet port and the outlet port are each an oval port, a teardrop-shaped port, a triangular port, or a plurality of adjacent port openings having different sizes.

In some examples, the inlet port and the outlet port each have a non-circular shape and the inlet opening and the outlet opening each have a generally circular shape.

In certain embodiments, the inlet port and the outlet port comprise a first port set, the hydraulic prosthetic further including at least one additional port set having an additional inlet port and an additional outlet port in the movable portion, the movable portion being selectively positionable in a first orientation, wherein the ports in the first port set are disposed adjacent the respective openings in the housing, and in a second orientation, wherein the ports in the at least one additional port set are disposed adjacent the respective openings in the housing, the first port set and the second port set providing different hydraulic tuning characteristics.

In some examples, the first one-way valve is a check valve disposed in the first fluid passage and the second one-way valve is a check valve disposed in the second fluid passage.

In certain embodiments, the prosthetic may include a force gauge, the force gauge having a Hall effect sensor and a magnet mounted on the prosthetic such that loading on the prosthetic causes a distance between the magnet and the Hall effect sensor to change, thus providing information on a force applied to the prosthetic.

According to a second embodiment, a prosthetic, with control of bidirectional movement, includes a prosthetic foot having an upper portion for connection to a leg and a lower foot portion movably connected to the upper portion. A hydraulic cylinder has a movable piston and a first chamber and a second chamber, the hydraulic cylinder coupled to the upper and lower portions of the prosthetic foot such that dorsiflexion of the foot causes the first chamber to reduce in size and the second chamber to expand and plantarflexion of the foot causes the first chamber to expand and the second camber to reduce in size. A three-way valve includes a housing and a movable portion that is movable relative to the housing, the housing and movable portion cooperating to define a hydraulic circuit. The movable portion has a master port, an inlet port and an outlet port, the master port being in fluid communication with the inlet and outlet ports. The housing has an inlet opening positioned to selectively fluidly communicate with the inlet port of the movable portion, an outlet opening positioned to selectively fluidly communicate with the outlet port of the movable portion, and a master opening positioned to fluidly communicate with the master port. A first fluid passage extends from the first chamber to the master opening, a second fluid passage extends from the second chamber to the inlet opening of the housing, and a third fluid passage extends from the second chamber to the outlet opening of the housing. The valve and fluid passages cooperating to selectively define a plantarflexion fluid circuit from the second chamber, through the second fluid passage, through the inlet opening and the inlet port, to and through the master port and master opening and through the first fluid passage to the first chamber of the hydraulic cylinder; and to selectively define a dorsiflexion circuit from the first chamber, through the first fluid passage, through the master opening and master port, to and through the outlet port and outlet opening, and through the third fluid passage to the second chamber of the second chamber of the hydraulic cylinder. A first one-way valve is disposed in the plantarflexion circuit operable to allow fluid to flow substantially only in a direction from the second chamber to the first chamber, and a second one-way valve is disposed in the dorsiflexion circuit operable to allow fluid to flow substantially only in a direction from the first chamber to the second chamber. The inlet port and/or the inlet opening, and the outlet port and/or the outlet opening are shaped such that movement of the movable portion of the valve relative to the housing varies a resistance to the fluid flow in the plantarflexion and in the dorsiflexion circuits.

A method of controlling bidirectional movement of a hydraulic prosthetic, is also provided by an embodiment of the present invention. The method includes providing a prosthetic having an upper portion and a lower portion movably connected to the upper portion; and providing a hydraulic cylinder having a movable piston and a first chamber and a second chamber, the hydraulic cylinder coupled to the upper and lower portions of the prosthetic such that movement of the lower portion relative to the upper portion in a first direction causes the first chamber to reduce in size and the second chamber to expand and movement of the lower portion relative to the upper portion in a second direction causes the first chamber to expand and the second camber to reduce in size; and providing a first fluid circuit from the first chamber to the second chamber and a second fluid circuit from the second chamber to the first chamber. The method further includes substantially blocking fluid flow through the first fluid circuit from the second chamber to the first chamber and substantially blocking fluid flow through the second fluid circuit from the first chamber to the second chamber, and controlling a resistance to fluid flow in the first fluid circuit and in the second fluid circuit using a three way valve, the valve having an inlet port and/or an inlet opening, and an outlet port and/or an outlet opening that are shaped such that movement of a movable portion of the valve relative to a housing of the valve varies a resistance to the fluid flow in both the first and second fluid circuits. In some examples, the method utilizes any of the embodiments of the prosthetic, cylinder and valve disclosed herein.

According to a further embodiment, a prosthetic foot includes an upper portion for connection to a leg and a lower foot portion movably connected to the upper portion. A Hall effect sensor is mounted on the prosthetic foot such that loading on the foot causes a distance between a magnet and a transducer of the Hall effect sensor to change, thus providing information of a force applied.

### BRIEF DESCRIPTION OF THE DRAWINGS

FIG. 1 is a perspective view of a prosthetic foot according to an embodiment of the present invention;

FIG. 2 is a longitudinal cross-sectional view of the foot of FIG. 1;

FIG. 3 is a rear perspective view of the foot of FIGS. 1 and 2 with portions cut away to reveal a three-way valve according to an aspect of the present invention;

FIG. 4 is a schematic showing an embodiment of a hydraulic cylinder, three-way valve and fluid passages, according to an aspect of the present invention;

FIG. 5 is a schematic showing a three-way valve according to an aspect of the present invention;

FIG. 6 is a perspective view of a movable portion of a three-way valve for use with certain embodiments of the present invention;

FIG. 7 is a cross-sectional view of the movable portion of FIG. 6;

FIG. 8 is a cross-sectional view of a portion of a prosthetic foot with the movable portion of FIG. 6 disposed therein;

FIG. 9 is a schematic showing a series of positions of a port relative to an opening to illustrate the change in flow area;

FIG. 10 is a schematic showing alternative port shapes;

FIGS. 11A-11C are schematics illustrating an example of valve positions during a standing support mode (11A), a stairs up/down mode (11B), and a walking mode (11C), with increased stability and high resistance, where plantarflexion resistance (P) is equal to dorsiflexion resistance (D);

FIGS. 12A-12C are schematics illustrating an example of valve positions during a ramp up mode or when a walking speed increases, with an easier forward motion, where P=D+3;

FIGS. 13A-11C are schematics illustrating an example of valve positions during a ramp down mode or a transition mode, with reduced speed and increased stability, where P=D−3; and

FIG. 14 is a perspective view of a hydraulic prosthetic knee that may form an embodiment of the present invention.

### DETAILED DESCRIPTION OF THE INVENTION

### Single Valve Control of Bidirectional Movement of Hydraulic Prosthetic Device

An embodiment of the present invention provides a control for movement in first and second directions of a hydraulic prosthetic device using a single three-way valve. An example of movement in a first and second direction is plantarflexion and dorsiflexion of a hydraulic prosthetic foot. The present invention is also applicable to other prosthetic devices such as, but not limited to, a prosthetic knee, where movement is to be controlled in two directions.

The three-way valve controls the resistance to flow of hydraulic fluid due to movement in the first direction and in the second direction, and allows the resistance to both fluid flows to be adjusted by moving the single three-way valve. Ports and/or openings in the movable portion and housing of the valve are shaped such that movement of the movable portion relative to the housing varies the resistance to both flows. A motor may control movement of the movable portion, and a plurality of port opening pairs may be provided to allow changes in the relative flow resistances. The present invention will be described with references to certain embodiments but is not limited to these embodiments.

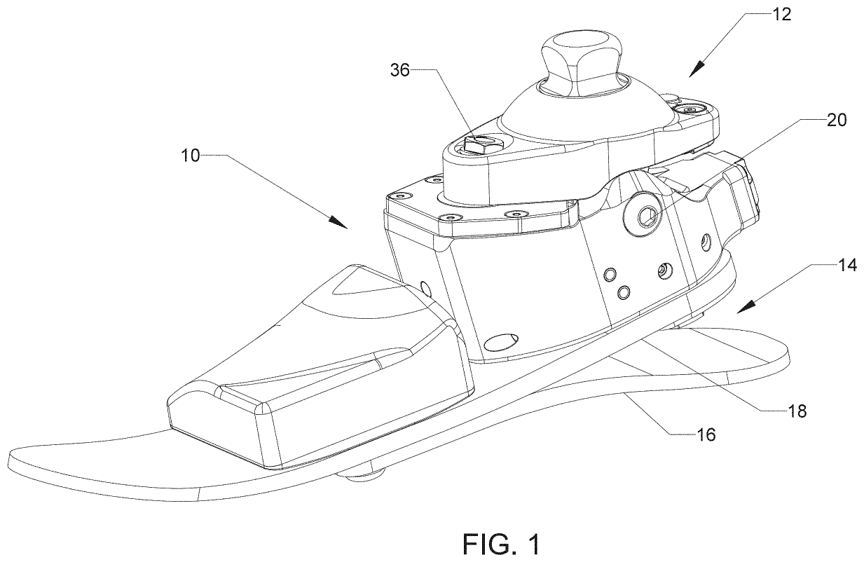

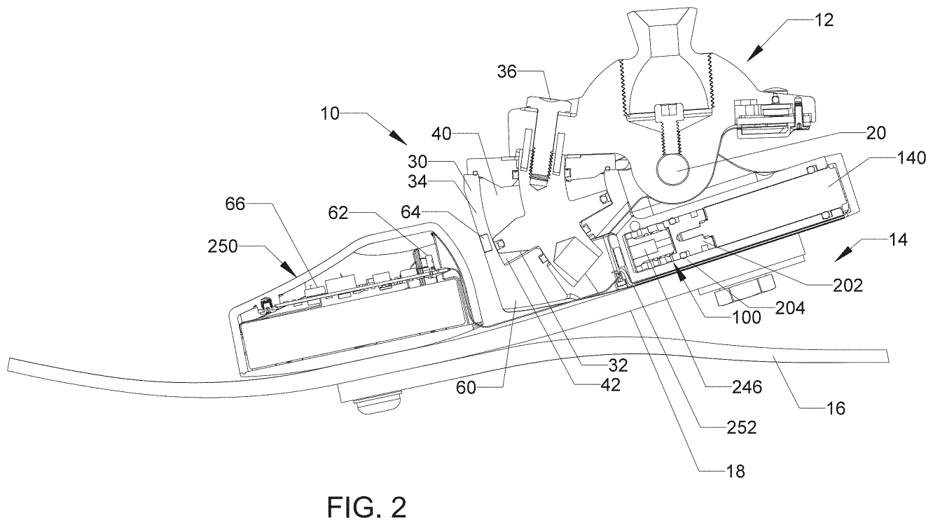

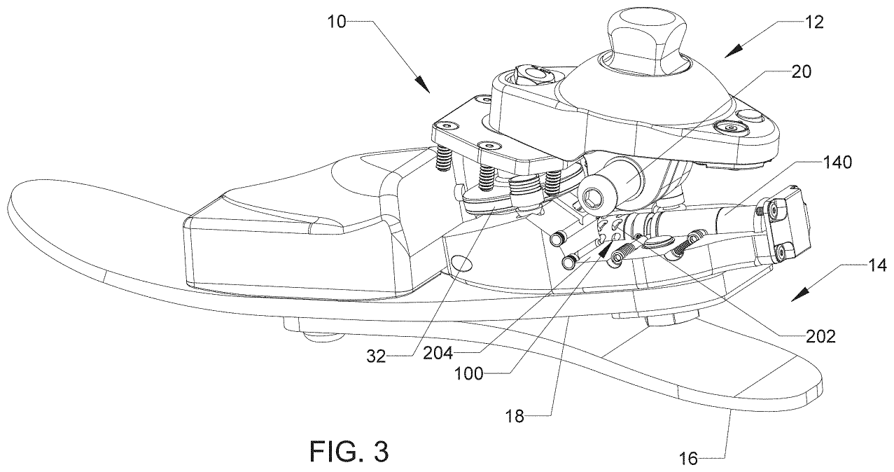

FIGS. 1-3 provide various views of an exemplary hydraulic prosthetic foot 10 which may form an embodiment of the present invention. FIG. 1 is a perspective view, FIG. 2 is a longitudinal cross-sectional view, and FIG. 3 is a rear perspective view with portions of the foot cut away to show a valve assembly. The foot 10 has an upper portion 12 with a pylon attachment for attaching to a lower leg prosthesis or adapter and a lower portion 14 defining a lower part of the foot. The lower portion 14 in this example includes a base spring 16 and a heel spring 18 to provide energy return. The upper portion 12 and lower portion pivot relative to each other about a pivot member 20, defining a pivot axis. The term “pivot” or “pivotally” as used herein encompasses a traditional pivot as well as flexing joints of various types that allow relative movement of parts.

A hydraulic cylinder is provided for damping the pivot motion of the lower portion 14 relative to the upper portion. In this example, the hydraulic cylinder is a curved hydraulic cylinder 30, shown in cross-section in FIG. 2. The hydraulic cylinder includes a double-sided piston 32 that is movable within a curved housing 34. A shaft of the piston is connected to the upper portion 12 by connection member 36 and the housing 34 is connected to the heel spring 18 such that pivotal movement between the upper portion 12 and lower portion 14 causes they piston 32 to move within the housing 34. A first chamber 40 is defined in the cylinder housing 34 above the piston 32 and a second chamber 42 is defined below the piston 32. During dorsiflexion, defined as the toe portion of the foot pivoting upwardly, the first chamber 40 is reduced in size and the second chamber 42 is expanded. During plantarflexion, defined as the toe portion of the foot pivoting downwardly, the first chamber 40 is expanded and the second chamber 42 is reduced in size. By controlling fluid flow between the first and second chambers, dorsiflexion and plantarflexion may be damped. Adjusting a resistance to the fluid flow will adjust the resistance to movement. The prosthetic may also be referred to as having movement of a lower portion relative to an upper portion in a first direction and a second direction. In one example, dorsiflexion is movement in the first direction and plantarflexion is movement in the second direction. The terms “upper portion” and “lower portion” are not limiting on the orientation of the various parts of the prosthesis but are used merely for convenience.

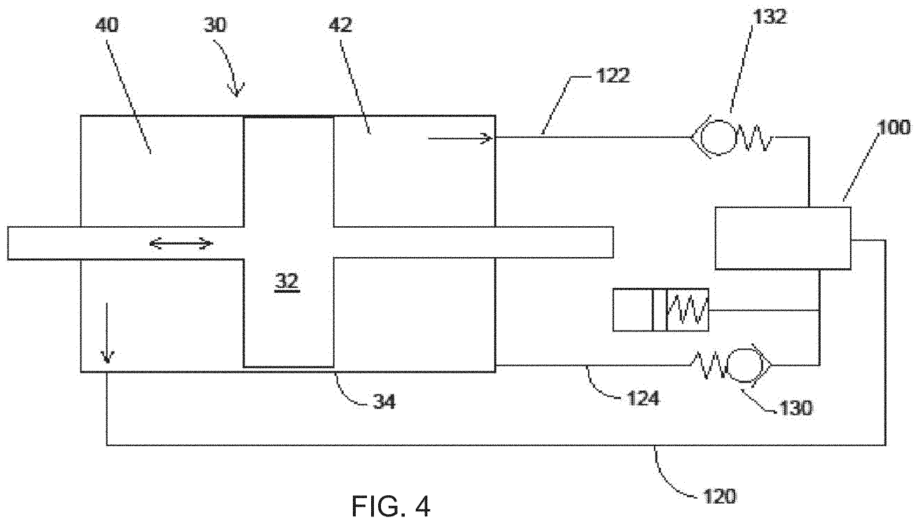

In the illustrated embodiment, resistance to fluid flow between the chambers 40 and 42 is controlled by a three-way valve 100 that is disposed aft of the hydraulic cylinder and is connected thereto by fluid passages. FIG. 4 provides a schematic illustration of the arrangement of the three-way valve and its connection to the cylinder. The same element numbers will be used but the schematic is not limited to the configuration of FIGS. 1-3.

The hydraulic cylinder 30 has a movable piston 32 in a housing 34 with a first chamber 40 defined on one side of the piston and a second chamber 42 defined on the other. While not illustrated in FIG. 4, the hydraulic cylinder is mechanically coupled to a hydraulic prosthetic device such that movement of one portion of the prosthetic device relative to another portion causes one of the chambers to expand in size and the other to reduce in size, or vice versa for movement in the opposite direction.

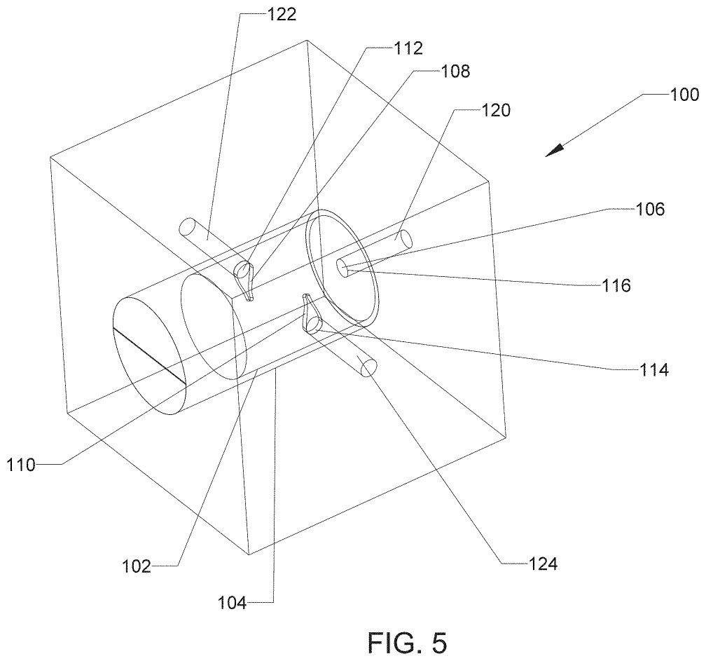

The three-way valve 100 is schematically shown in FIG. 5. The valve 100 has a movable portion 102 disposed in a housing 104, which are illustrated as nested cylinders. In some examples, the three-way valve is a rotary spool valve, though those of skill in the art will recognize that the invention may be practiced with other valve designs having a housing with a movable portion that can be moved relative to the housing, such as in a linear or rotary motion. The movable portion 102 and the housing 104 cooperate to define a hydraulic circuit. The movable portion has a master port 106, which is illustrated as an opening in an end wall of the cylindrical movable portion 102, but may be formed in various ways. The movable portion further has an inlet port 108 and an outlet port 110, which are illustrated as diametrically opposed teardrop-shaped openings in the sidewall of the cylindrical movable portion, but may also be formed in various ways. The master port 106 is in fluid communication with the inlet port 108 and outlet port 110 so that fluid that flows in one of the ports may flow out the others, and vice versa.

The housing 104 has fluid passages and openings positioned to fluidly communicate with the ports of the movable portion. The housing has an inlet opening 112 positioned to selectively fluidly communicate with the inlet port 108 of the movable portion. The inlet opening 112 is represented by a circular opening in FIG. 5, but it may have other shapes. The housing also has an outlet opening 114 positioned to selectively fluidly communicate with the outlet port 110. The outlet opening 114 is represented by a circular opening in FIG. 5 but may have other shapes. The housing also has a master opening 116 positioned to fluidly communicate with the master port 106. The master opening 116 is represented by a circular opening in FIG. 5 but may have other shapes. The term “selectively” is used herein to indicate that the fluid communication may be blocked in some positions, such that the ability to communicate is selected during operation.

Fluid passages extend between the openings in the housing 104 and the chambers of the hydraulic cylinder, and cooperate with the valve to define fluid circuits. A first fluid passage 120 extends from the first chamber 40 to the master opening 116. The first fluid passage 120 is shown schematically in FIG. 4 and a portion of the passage is shown schematically in FIG. 5. A second fluid passage 122 extends from the second chamber 42 to the inlet opening 112. A third fluid passage 124 extends from the second chamber 42 to the outlet opening 114.

The fluid circuits are defined as follows. A first fluid circuit extends from the first chamber 40, through the first fluid passage 120, through the master opening 116 and master port 106, to and through the outlet port 10 and outlet opening 114, and through the third fluid passage 124 to the second chamber 42 of the hydraulic cylinder 30. A first one-way valve 130, which may be a check valve, is disposed in the first fluid circuit and is operable to allow fluid to flow substantially only in a direction from the first chamber to the second chamber. Therefore, the first fluid circuit carries fluid from the first chamber 40 to the second chamber 42 during movement in the first direction, which may be dorsiflexion.

A second fluid circuit extends from the second chamber 42, through the second fluid passage 122, through the inlet opening 112 and the inlet port 108, to and through the master port 106 and master opening 116 and through the first fluid passage 120 to the first chamber 40 of the hydraulic cylinder 30. A second one-way valve 132, which may be a check valve, is disposed in the second fluid circuit and is operable to allow fluid to flow substantially only in a direction from the second chamber to the first chamber. Therefore, the second fluid circuit carries fluid from the second chamber 42 to the first chamber 40 during movement in the second direction, which may be plantarflexion. The phrase “substantially only in a direction” means that the valve resists flow in an opposite direction but does not require an absolute seal, as long as the function of the hydraulic system is maintained.

As will be clear to those of skill in the art, resistance to movement of the hydraulic cylinder in the first and second direction will depend on the resistance to flow in the first and second fluid circuits. In embodiments of the present invention, the inlet port 108 and/or the inlet opening 112, and the outlet port 110 and/or the outlet opening 114 are shaped such that movement of the movable portion 102 of the valve 100 relative to the housing 104 varies a resistance to the fluid flow in both the first and second fluid circuits. FIG. 5 illustrates this schematically. The inlet port 108 and the outlet port 110 are each teardrop shaped, with a wider end and a narrower end, while the inlet opening 112 and the outlet opening 114 are round. By rotating the movable portion 102 relative to the housing 104, the portion of the port which aligns with the respective opening will transition from large, at the large end of the port, to small, at the small end of the port. This changes the resistance from high to low. The ports and openings are both shown in a low resistance position in FIG. 5.

It is noted that the ports and openings are referred to as inlet or outlet ports or openings, to reflect the direction of flow through those particular ports or openings in these embodiments, but such terms are not limiting on the invention.

Certain embodiments of the present invention use a three way valve with a plurality of port pairs to allow the relative resistance to flow to be chosen. FIGS. 2 and 3 illustrate such an embodiment, wherein a valve 100 has a rotationally movable portion 202 disposed in a housing 204 defined by the body of the lower portion 14 of the prosthetic foot. In this embodiment, a motor 140 engages the movable portion 202 and is operable to rotate the movable portion.

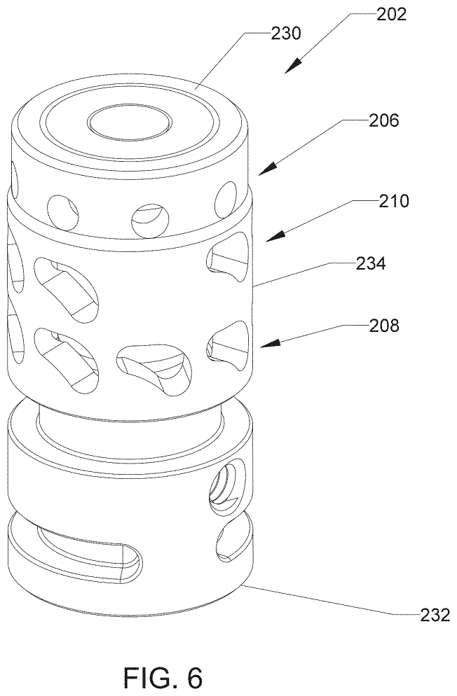

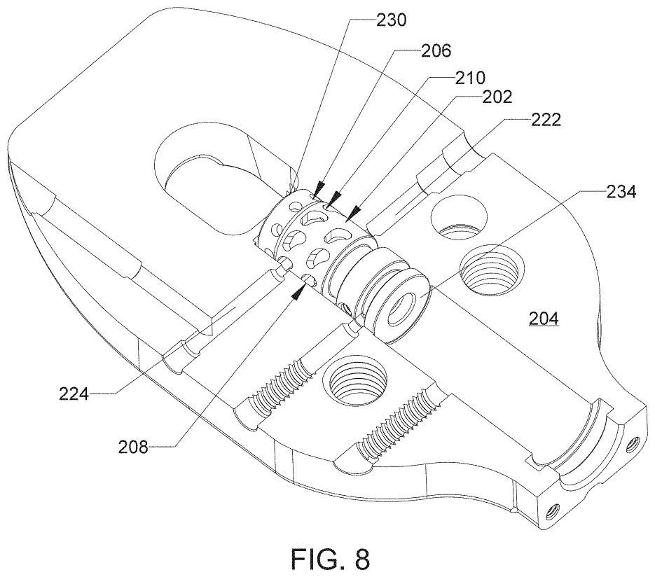

FIGS. 6-8 provide more detailed views of the movable portion 202, with FIG. 8 also showing a cut-away portion of the housing 204. The movable portion 202 is generally cylindrical with a first end surface 230, and opposite second end surface 232 and a circumferential side surface 234 extending between the end surfaces. The inlet ports, outlet ports and a plurality of master ports are defined in the side surface. In this example, a plurality of inlet ports 208 are provided in a first row, a plurality of outlet ports 210 are provided in a second row, and a plurality of master ports 206 are provided in a third row, with the rows arranged side by side along the length of the side surface 234. As shown in FIG. 8, the row of inlet ports 208 are aligned with the second fluid passage 222 and the row of outlet ports 210 are aligned with the third fluid passage 224. The passages 222 and 224 are diametrically opposed and offset laterally. A first fluid passage, not shown, communicates with the master ports 206. In some examples, the side surface in the area where the master ports are provided is slightly smaller in diameter than the housing 204 in this area such that a circumferential passage extends around the perimeter to allow communication between the first fluid passage and the master ports independent of rotational orientation.

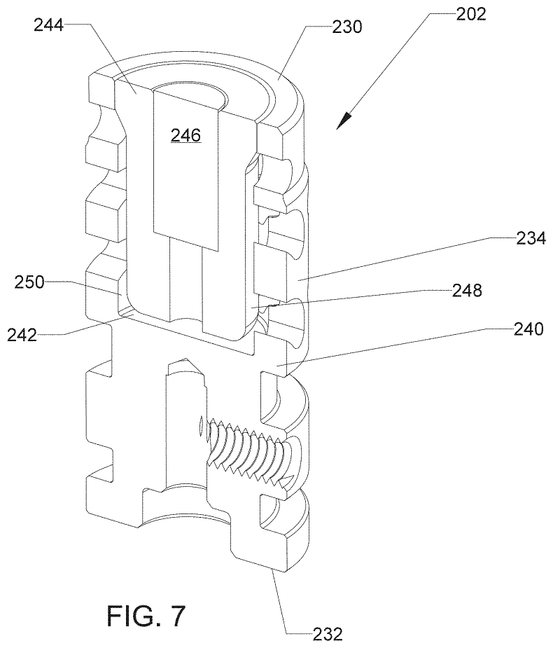

Referring now to FIG. 7, the movable part 202 has an outer body 240 with a recess 242 extending inwardly from the first end surface 230. An insert 244 is inserted into the recess 242 and holds a magnet 246, which may be used for rotational encoding of the position of the movable portion 202. The insert 244 and magnet 246 seal the end surface 244. An outer surface 248 of the insert is spaced from the inner surface 250 so as to provide an annular chamber extending between all of the ports 206-210.

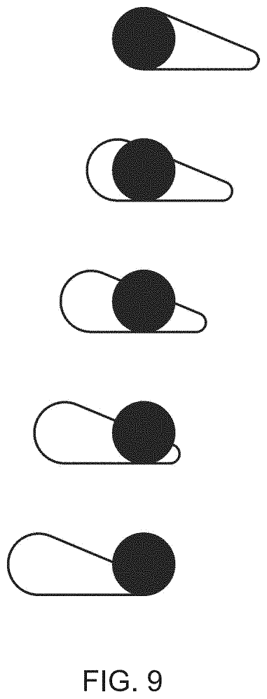

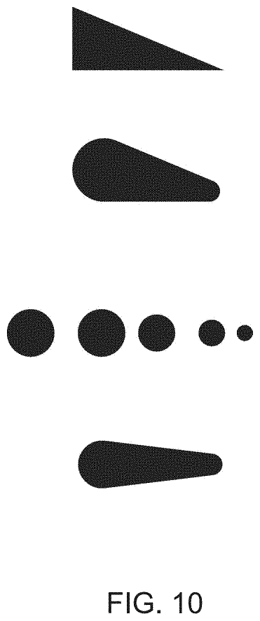

As mentioned, a motor 140 may be provided for rotating the movable portion 202. In some examples, the motor may rotate the movable portion when the foot is in a swing phase, when no loading is provided, thereby reducing the load on the motor and valve. This helps to allow the use of a small motor, and reduces power requirements. Referring again to FIG. 2, a control 250 may be provide in the foot, including a control circuit and a power supply, such as a battery. The control powers and controls the motor. A rotational encoder 252 may be provided adjacent the magnet 246 for sensing the position of the movable portion, to allow closed-loop control. As will be clear to those of skill in the art, the rotation of the movable portion in the housing alters the resistance to fluid flow, depending on where the opening lines up with the port. The arc length of the ports dictates how much angular adjustment the movable portion will have in for a particular port set. In some embodiments, such as illustrated, the ports take a shape of a teardrop. FIG. 9 schematically illustrates how the flow area between a port and opening varies with relative position. Other shapes of ports can be used, such as triangular, oval, or kidney shaped, as long as the rotation of the ports relative to the fixed passages would vary the amount of oil flow. FIG. 10 illustrates a triangular opening, a teardrop shaped opening, a plurality of adjacent port openings having different sizes, and a generally oval or elongated opening. An ellipsoidal opening is also possible. For the purposes of this disclosure, for certain embodiments, the series of adjacent port openings having different sizes may be considered a single “port” while in other embodiments only a continuous, shaped port is considered a port.

The single valve control in accordance with an embodiment of the present invention requires only one motor used to turn the movable portion at each state change. The movable portion may be fixed at each position unless the motor physically moves it. Having a single motor increases the battery life. In some examples, a port may have an adjustment range of 30 degrees from fully open to fully restricted. In one example, each port set has 10 positions or 1 position every 3 degrees.

Since a single valve is used to control oil flow in two directions, both the plantar and dorsiflexion resistance are dependent on each other. In some examples, the resistance to flow for each port is equal, such that both are fully open or fully restricted, depending on the position of the movable portion. As will be clear to those of skill in the art, it may be desirable to change the relationship between the resistance levels, such that one flow direction is always more restricted than the other, by a various amounts, or other relationships. The plurality of ports shown in FIGS. 6-8 allow for various combinations. These may be referred to as port sets, with each set including an inlet port and an outlet port. In the illustrated embodiment, the port sets are offset rotationally from each other. The motor may reposition the movable portion to utilize different port sets for different conditions, with adjustment being manually made or controlled by the control.

Various alternatives will not be described with respect to dorsiflexion resistance (D) and plantarflexion resistance (P). In some embodiments, the port sets can be configured such that P is an inverse of D, or P=D+1, or P=D+2, or P=D−1, or P=D−2 and so on, with the units of “1,” “2,” etc. depending on the application.

Based on the real data collected, it is determined that a correlation exists between plantarflexion (P) and dorsiflexion (D). The setting for P is typically within 3 units of D. Therefore, an embodiment may have 7 predetermined resistance ranges. Positions of the oil passages are adjustable within each range. Based on the formula P=D+X, seven unique side port profiles are used in this embodiment: P=D−3, P=D−2, P=D−1, P=D, P=D+1, P=D+2, P=D+3. Different environmental conditions require certain resistance settings for both plantar and dorsiflexion. Four to five port sets may be required to adequately meet the design requirements of valve positions for activities like walking, running, stability, and up and down hill walking. If required and as space allows, more port sets can be added circumferentially around the valve.

Table 1 below provides an example of the valve positions corresponding to the relative relations between P and D.

TABLE 1

| | | |
|---|---|---|
| Standing support | Increased stability, high | P = D |
| Stairs up/down (stability) | resistance | |
| Walking mode | Prosthetist Defined | P = D (+/−2) |
| Ramp up (assist) | Easier forward motion | P = D + 3 |
| Walking speed increase | | |
| Ramp down (brake) | Reduced speed and | P = D − 3 |
| Transition | increased stability | |

Table 2 below provides an example showing detailed valve positions for hydraulic resistance rated from 1-10.

TABLE 2

| | | | |
|---|---|---|---|
| Standing support | Near lock high | PF = 9 | P = D |
| | resistance | DF = 9 | |
| Stairs up/down | Increased stability, | PF = 8 | P = D |
| (stability) | high resistance | DF = 8 | |
| Walking mode | Comfortable rate | PF = 5 | P = D |
| | walking, patient | DF = 5 | |
| | defined | | |
| Ramp up (assist) | Designed for easier | PF = 7 | P = D + 3 |
| | forward motion | DF = 4 | |
| Walking speed | Easier forward | PF = 8 | P = D + 4 |
| increase | motion | DF = 4 | |
| Ramp down (brake) | Reduced speed and | PF = 3 | P = D − 4 |
| | increased stability, | DF = 7 | |
| | faster foot flat | | |
| Transition | Reduced speed and | PF = 4 | P = D − 3 |
| | increased stability | DF = 7 | |

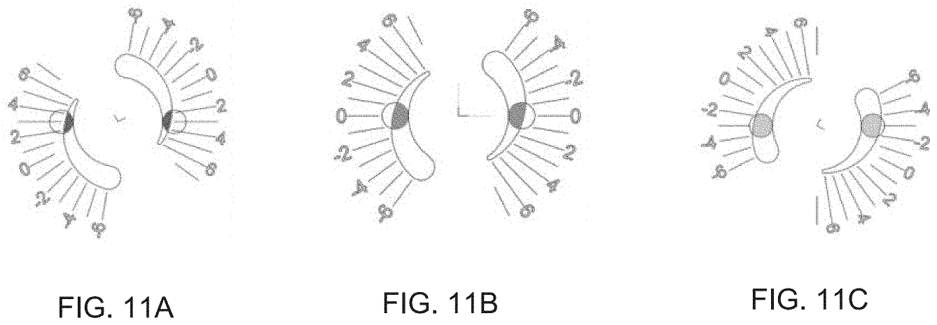

FIGS. 11A-11C schematically illustrate an example of valve positions during a standing support mode (11A), a stairs up/down mode (11B), and a walking mode (11C), with increased stability and high resistance, where P is equal to D. The valve positions are illustrated projected onto a flat surface but may be translated to the side surfaces of the rotationally movable portion.

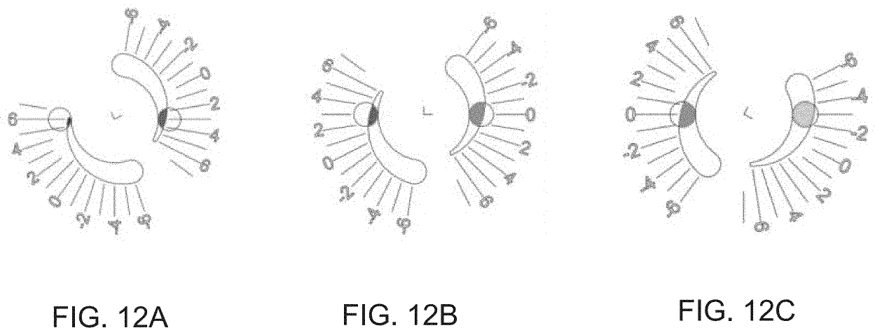

FIGS. 12A-12C illustrate an example of valve positions during a ramp up mode or when a walking speed increases, with an easier forward motion, where P=D+3.

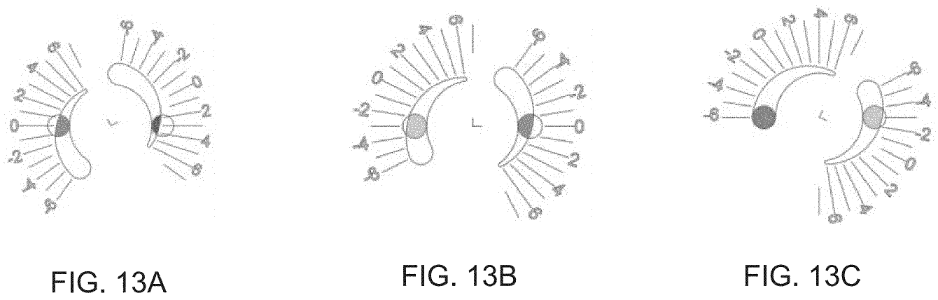

FIGS. 13A-11C illustrate an example of valve positions during a ramp down mode or a transition mode, with reduced speed and increased stability, where P=D−3.

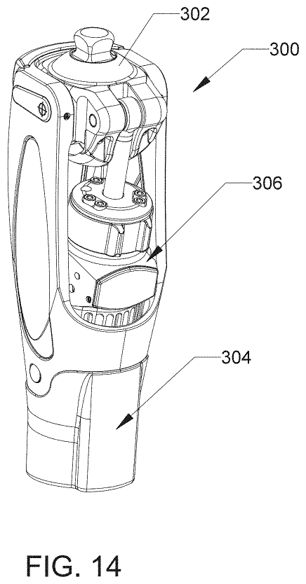

FIG. 14 provides a perspective view of a prosthetic knee 300 having an upper portion 302 movable relative to a lower portion 304 and a hydraulic cylinder 306. The present invention may take the form of a prosthetic knee, with the three-way valve controlling fluid resistance for the hydraulic cylinder. The present invention may also be used in other prosthesis.

Those of skill in the art will recognize that the embodiments described above may be altered in various ways without departing from the scope of the present invention. As one example, the openings in the housing could have a non-round shape, while the ports are non-round or round, but non-round ports in combination with round openings in the housing is generally preferred. As a further example, the generally cylindrical movable portion of the valve could have other shapes, including, but not limited to, a frustoconical shape.

### Hall Effect Sensor for Force Measurement

An embodiment of the present invention provides a method using a non-contact means for determining loading on a prosthetic foot, such as foot 10. As will be clear from FIG. 2, the base spring 16 and heel spring 18 deflect to provide energy return. The springs, in concert with the housing and the upper portion, make up the basic mechanical structure of the foot. The springs and housing 60 move relative to one another as the user goes through a gait cycle.

The bending of the base spring 16 is proportional to the force applied. Therefore, the distance between the housing and spring can be used as a measure of the applied force. A Hall sensor may be used to measure the force applied. The relationship between the bending of the base spring and the force applied is non-linear, and related to the dimensions of the base, as well as material choices. The deflection will also depend on where a measurement is taken. The different patient weight ranges are managed by different size springs and weight categories. The non-contact nature of a Hall sensor means that its use as a force gauge cannot be damaged by excessive force or travel of the springs.

FIG. 2 illustrates an example wherein a Hall effect sensor 62 is provided inside the control housing 250 and senses the relative movement of a magnet 64 disposed in the housing 60. This movement is proportional to the bending of the springs. Alternatively, the Hall effect sensor and magnet may be located different. The system may also include a multi-axis inertial measurement unit (IMU) such a 9 axis IMU. The IMU may be enclosed in a waterproof enclosure such as the control enclosure. A user interface may also be provided. The control may include any necessary circuitry.

In a Hall effect device, the signal output is proportional to field strength, which drops off as the square of the distance to the magnet. Different magnet shapes present different field strengths or shapes. In one example, the use of a cylindrical shape, with the poles at opposite ends, makes for a longer reach of the magnetic field and provides a greater working distance or analog signal range for the Hall sensor. Ideally, the range of the sensor exceeds the travel distance between the magnet and sensor. As known to those of skill in the art, the Hall sensor output versus distance to the sensor relationship is a curve, but can be approximated to a linear relationship over a smaller range. The saturation point of this combination is reached when the magnet gets too close, but the system may be designed so as to provide information over the range of typical use. As the magnet gets closer, eventually the signal reaches a limit and remains flat. Each sensor has an optimal range, and once exceeded, it will exhibit a rail effect. On the opposite end, the curve will approach or be asymptotic to a minimum value where no field is within range. The sensor is insensitive at the greater distances compared to the shorter distances, therefore the maximum output side of the curve should be used.

The robust nature of the sensor, as well as the large effective range, makes for an ideal sensing solution. Patients can be highly abusive of their prosthetic devices, usually without being aware of the forces that are being applied.

In one embodiment, the gap between the magnet and the sensor is set up such that the distance between the magnet and sensor can grow as the flexing member bends in the opposite direction. This negative load or pulling effect can occur in a strain gauge as well, resulting in deflection of the resistance in the opposite direction. In the gait cycle and with a composite mechanical foot, this occurs as the user steps on the foot base. Then as the foot is loaded, the flex is primarily a bend of the toe section as the user passes mid-stance.

In practice, the Hall force gauge is an excellent facsimile to a strain gauge, with no attempt to adjust the output or apply a transform to increase linearity. Alternatively, the output may be transformed for use as an input to other systems. A calibration curve may be used, and such a curve may be patient specific, set at the factory, or determined by a prosthetist.

The present invention has been described with reference to some embodiments. However, it is realized that variants and equivalents to the preferred embodiments may be provided without departing from the scope of the invention as defined in the accompanying claims. It is to be understood that both the foregoing general description and the following detailed description of the present invention are exemplary and explanatory and are intended to provide further explanation of the invention as claimed. It is not intended to be exhaustive or to limit embodiments to the precise form disclosed. It is the following claims, including all equivalents, which define the scope of the present invention.

1. A hydraulic prosthetic with control of bidirectional movement, comprising:
a prosthetic having an upper portion and a lower portion movably connected to the upper portion;
a hydraulic cylinder having a movable piston and a first chamber and a second chamber, the hydraulic cylinder coupled to the upper and lower portions of the prosthetic such that movement of the lower portion relative to the upper portion in a first direction causes the first chamber to reduce in size and the second chamber to expand and movement of the lower portion relative to the upper portion in a second direction causes the first chamber to expand and the second camber to reduce in size;
a three-way valve comprising;
a housing and a movable portion that is movable relative to the housing, the housing and movable portion cooperating to define a hydraulic circuit;
the movable portion having a master port, an inlet port and an outlet port, the master port being in fluid communication with the inlet and outlet ports;
the housing having an inlet opening positioned to selectively fluidly communicate with the inlet port of the movable portion, an outlet opening positioned to selectively fluidly communicate with the outlet port of the movable portion, and a master opening positioned to fluidly communicate with the master port;
a first fluid passage extending from the first chamber to the master opening;
a second fluid passage extending from the second chamber to the inlet opening of the housing;
a third fluid passage extending from the second chamber to the outlet opening of the housing;
the valve and fluid passages cooperating to selectively define;
a first fluid circuit from the first chamber, through the first fluid passage, through the master opening and master port, to and through the outlet port and outlet opening, and through the third fluid passage to the second chamber of the hydraulic cylinder;
a second fluid circuit from the second chamber, through the second fluid passage, through the inlet opening and the inlet port, to and through the master port and master opening and through the first fluid passage to the first chamber of the hydraulic cylinder; and
a first one-way valve disposed in the first fluid circuit operable to allow fluid to flow substantially only in a direction from the first chamber to the second chamber; and
a second one-way valve disposed in the second fluid circuit operable to allow fluid to flow substantially only in a direction from the second chamber to the first chamber;
the inlet port and/or the inlet opening, and the outlet port and/or the outlet opening being shaped such that movement of the movable portion of the valve relative to the housing varies a resistance to the fluid flow in both the first and second fluid circuits.

2. The hydraulic prosthetic according to claim 1, wherein the prosthetic comprises a prosthetic foot, the upper portion being for connection to a leg and the lower portion being a lower foot portion, the movement in the first direction being dorsi-flexion and the movement in the second direction being plantarflexion.

3. The hydraulic prosthetic according to claim 1, further comprising a motor operable to move the movable portion of the valve relative to the housing for adjusting the resistance to fluid flow in the circuits.

4. The hydraulic prosthetic according to claim 1, wherein the three way valve is a spool valve with the movable portion being rotatably movable relative to the housing.

5. The hydraulic prosthetic according to claim 4, wherein the movable portion has an end surface and a circumferential side surface, the master port, inlet port, and outlet port being disposed in the side surface.

6. The hydraulic prosthetic according to claim 5, wherein the inlet port and outlet port and disposed on opposite sides of the movable portion.

7. The hydraulic prosthetic according to claim 1, wherein the inlet port and the outlet port each comprise:
an oval port;
a teardrop-shaped port;
a triangular port; or
a plurality of adjacent port openings having different sizes.

8. The hydraulic prosthetic according to claim 1, wherein the inlet port and the outlet port each have a non-circular shape and the inlet opening and the outlet opening each have a generally circular shape.

9. The hydraulic prosthetic according to claim 1, wherein the inlet port and the outlet port comprise a first port set, the hydraulic prosthetic further comprising at least one additional port set having an additional inlet port and an additional outlet port in the movable portion, the movable portion being selectively positionable in a first orientation, wherein the ports in the first port set are disposed adjacent the respective openings in the housing, and in a second orientation, wherein the ports in the at least one additional port set are disposed adjacent the respective openings in the housing, the first port set and the second port set providing different hydraulic tuning characteristics.

10. The hydraulic prosthetic according to claim 1, wherein the first one-way valve is a check valve disposed in the first fluid passage and the second one-way valve is a check valve disposed in the second fluid passage.

11. The hydraulic prosthetic according to claim 1, further comprising a force gauge, the force gauge having a Hall effect sensor and a magnet mounted on the prosthetic such that loading on the prosthetic causes a distance between the magnet and the Hall effect sensor to change, thus providing information on a force applied to the prosthetic.

12. A prosthetic foot with control of bidirectional movement, comprising:
a prosthetic foot having an upper portion for connection to a leg and a lower foot portion movable connected to the upper portion;
a hydraulic cylinder having a movable piston and a first chamber and a second chamber, the hydraulic cylinder coupled to the upper and lower portions of the prosthetic foot such that dorsiflexion of the foot causes the first chamber to reduce in size and the second chamber to expand and plantarflexion of the foot causes the first chamber to expand and the second camber to reduce in size;
a three-way valve comprising;
a housing and a movable portion that is movable relative to the housing, the housing and movable portion cooperating to define a hydraulic circuit;
the movable portion having a master port, an inlet port and an outlet port, the master port being in fluid communication with the inlet and outlet ports;
the housing having an inlet opening positioned to selectively fluidly communicate with the inlet port of the movable portion, an outlet opening positioned to selectively fluidly communicate with the outlet port of the movable portion, and a master opening positioned to fluidly communicate with the master port;
a first fluid passage extending from the first chamber to the master opening;
a second fluid passage extending from the second chamber to the inlet opening of the housing;
a third fluid passage extending from the second chamber to the outlet opening of the housing;
the valve and fluid passages cooperating to selectively define;
a plantarflexion fluid circuit from the second chamber, through the second fluid passage, through the inlet opening and the inlet port, to and through the master port and master opening and through the first fluid passage to the first chamber of the hydraulic cylinder; and
a dorsiflexion circuit from the first chamber, through the first fluid passage, through the master opening and master port, to and through the outlet port and outlet opening, and through the third fluid passage to the second chamber of the second chamber of the hydraulic cylinder;
a first one-way valve disposed in the plantarflexion circuit operable to allow fluid to flow substantially only in a direction from the second chamber to the first chamber; and
a second one-way valve disposed in the dorsi-flexion circuit operable to allow fluid to flow substantially only in a direction from the first chamber to the second chamber;
the inlet port and/or the inlet opening, and the outlet port and/or the outlet opening being shaped such that movement of the movable portion of the valve relative to the housing varies a resistance to the fluid flow in the plantarflexion and in the dorsiflexion circuits.

13. A method of controlling bidirectional movement of a hydraulic prosthetic, comprising:
providing a prosthetic having an upper portion and a lower portion movably connected to the upper portion;
providing a hydraulic cylinder having a movable piston and a first chamber and a second chamber, the hydraulic cylinder coupled to the upper and lower portions of the prosthetic such that movement of the lower portion relative to the upper portion in a first direction causes the first chamber to reduce in size and the second chamber to expand and movement of the lower portion relative to the upper portion in a second direction causes the first chamber to expand and the second camber to reduce in size;
providing a first fluid circuit from the first chamber to the second chamber and a second fluid circuit from the second chamber to the first chamber;
substantially blocking fluid flow through the first fluid circuit from the second chamber to the first chamber;
substantially blocking fluid flow through the second fluid circuit from the first chamber to the second chamber
controlling a resistance to fluid flow in the first fluid circuit and in the second fluid circuit using a three way valve, the valve having an inlet port and/or an inlet opening, and an outlet port and/or an outlet opening that are shaped such that movement of a movable portion of the valve relative to a housing of the valve varies a resistance to the fluid flow in both the first and second fluid circuits.

14. A prosthetic foot, comprising:
an upper portion for connection to a leg and a lower foot portion movably connected to the upper portion;
a Hall effect sensor mounted on the prosthetic foot such that loading on the foot causes a distance between a magnet and a transducer of the Hall effect sensor to change, thus providing information of a force applied.
